(19) United States
(12) Patent Application Publication
(10) Pub. No.: US 2010/0076261 A1
(43) Pub. Date: Mar. 25, 2010

(54) EXAMINATION DEVICE

(75) Inventors: Uri Neeman, Rishon Lezion (IL); Amos Neeman, Moshav Bazra (IL); Gershon Goldenberg, Carcur (IL)

Correspondence Address: Pearl Cohen Zedek Latzer, LLP, 1500 Broadway, 12th Floor, New York, NY, 10036, US

(73) Assignee: MEDVISION INC., Carcur (IL)

(21) Appl. No.: 12/442,999

(22) PCT Filed: Sep. 25, 2007
(86) PCT No.: PCT/IL07/01190

§ 371 Date: Nov. 30, 2009

Related U.S. Application Data
(60) Provisional application No. 60/847,648, filed on Sep. 28, 2006.

(51) Int. Cl. A61B 1/04; A61B 1/307; A61B 1/06

(52) U.S. Cl. 600/109; 600/114; 600/178

(57) ABSTRACT

A device for imaging a bladder, comprising an image sensor, an illumination source and a device orientation actuator.

### RELATED APPLICATIONS

This application claims the benefit of U.S. Provisional Patent Application No. 60/847,648, filed Sep. 28, 2006, which is incorporated herein by reference.

### FIELD OF THE DISCLOSURE

The present disclosure generally relates to a bladder examination device.

### BACKGROUND

The bladder is a part of the urinary system, which is a hollow muscular organ in the form of a balloon, located in the pelvic region. It stores the urine (which is composed of urea, water and other waste substances), which has been removed from the blood, filtrated in the kidneys and then deposited in the bladder. The bladder has the ability to stretch and to shrink, depending on the amount of stored urine. The environment inside the bladder is quite abrasive. The pH-value inside the bladder can range between about 5 and 8 pH. The pH-value indicates if a substance is acidic or neutral or basic/alkaline. If the pH-value is 7, than the substance is neutral, in other words basic and acid parts of the substance are well-balanced. Substances below the pH-value of 7 are acidic and substances above 7 are basic. Transitional cells (also called epithelial cells) line the bladder.

Bladder cancer can be a life-threatening disease if it is not detected and effectively treated at a sufficiently early stage. The incidence rate of bladder cancer, which may be defined as the number of new cases diagnosed per 100,000 persons per year, indicates that bladder cancer is the fourth most common type of cancer in men (after prostate, lung and colon) accounting for approximately 6.2% of all cancer cases, and the eighth most common type of cancer in females, accounting for approximately 2.5% of all cancer cases. In the USA alone approximately 53,000 new cases are diagnosed each year. Bladder cancer is more common in males than females by a ratio of 3 to 1. Males have higher survival rates over a period of 5 years, as compared to females. More information about bladder cancer may be found at http://en.wikipedia.org/wiki/Cancer and in United States, National Cancer Institute, A snapshot of bladder cancer, August 2005<http://planning.cancer.gov/disease/Bladder-Snapshot.pd, both herein incorporated by reference.

The most common type of bladder cancer is transitional cell carcinoma (TCC). Tumors originate within the transitional cells. A tumor limited to the transitional cells of the bladder is usually considered a superficial bladder cancer. Where the tumor reaches the stage where it grows through the bladder's inner surface and into the muscular wall of the bladder, it may be referred to as invasive bladder cancer. This form of cancer can spread to adjacent organs.

Therefore, in order to prevent the spread of the cancer, it is important that an examination be conducted to accurately determine whether various cancers or tumors may be present within or on the wall of the bladder. There are various diagnostic to methods currently in use such as cystoscopy (examination by means of a cytoscope—a tubular instrument equipped with a light source to examine the interior of the bladder and ureter), Ultrasound (US), computed tomography (CT), intravenous pyelography (IVP) (which is an X-ray imaging method of the urinary tract), ultrasound imaging of the urinary tract, as well as urine markers.

The purpose of cystoscopy is to visualize and inspect the bladder and the urethra. Cystoscopy employs insertion of an optic fiber (cystoscope) through the urethra and into the bladder in order to observe the bladder wall by use of an external video screen. The cystoscope is inserted through the urethra into the bladder under either full, spinal or local anesthesia. A sterile liquid flows through the cystoscope filling the bladder and stretching it to allow a view of the bladder wall. The duration of the procedure is 20-30 minutes in most cases. The existing cystoscopes are either rigid (a solid straight stiff fiber-optic telescope with a high intensity light source) or flexible (a pliable fiber-optic instrument with a maneuverable tip).

A problem with cystoscopy is that, due to difficulties in navigating the head of the optic fiber, the ability to explore the entire bladder wall is limited. A further disadvantage is that insertion of the optic fiber into the bladder via the urethra is accompanied with pain both during the procedure as well as afterwards. A further disadvantage is that despite the fact that a sterile liquid flows through the cystoscope and fills the bladder, there still remains the risk of infection. The required anesthesia for the examination is an additional health risk, which is especially relevant concerning elderly, patients.

Computed tomography (CT) is used for determining the stage (distant metastasis, enlarged lymph node) of the cancer but may not be able to detect bladder tumors that are smaller that 5 mm-10 mm. Nor can a computed tomography scan determine the exact nature of a bladder tumor.

Ultrasound imaging of the urinary tract is less sensitive than a CT in detecting bladder tumors. Ultrasound cannot assess distant metastasis or lymph node involvement. It is usually used as a primary imaging modality for investigation of macroscopic hematuria.

Malignant cells can be observed on microscopic examination of the urinary sediment. Urine cytology (the study of the microscopic appearance of cells, especially for diagnosis of abnormalities and malignancies) is more sensitive in patients with high grade tumors. However, even this method of detection has a false negative rate of 20% in high grade tumors.

An additional method of diagnosis of bladder cancer is through resection of the tumor and histological (tissue) analysis in order to determine the stage and grade of the tumor. Transurethral resection of a tumor (TURbT) is both a diagnostic and therapeutic modality. It is estimated that in 30-45% of resections a residual tumor is overlooked; therefore, a second TURbT is mandatory in most cases of high grade superficial tumors.

After resection of the primary bladder tumor, recurrence of bladder cancer is estimated at 70% and progression to invasive bladder cancer 15-40%. Therefore, regular monitoring of the bladder is indicated, with periodic cystoscopy direct imaging.

Superficial bladder cancer is usually treated with methods aimed at preserving the bladder, and periodically followed up to diagnose recurrence of bladder cancer. Invasive bladder cancer usually requires a more aggressive treatment, usually a cystectomy (surgical extraction of the entire bladder).

The primary treatment of superficial bladder cancer is TURbT. TURbT is primarily diagnostic and performed after cystoscopic visualization of a bladder tumor has been done. While resection of the tumor should be as thorough as possible, so that all of the tumor may be removed, as previously mentioned, residual tumor is diagnosed in 30-45% of cases of TURbT. Therefore, a second cystoscopy is normally performed 2-4 weeks after the primary resection. After the tumor is examined by the pathologist, staging and grading of the tumor is made.

Superficial low grade tumors require no additional treatment and are managed by periodic cystoscopies. Superficial high grade tumors require additional treatment by administration into the bladder of immunotherapeutic or chemotherapeutic agents. Bacillus Calmette-Guerin (BCG) may be used as an immunotherapeutic agent. BCG is derived from living bacteria (germs) similar to TB (tuberculosis). Intensive cystoscopic follow-up is mandatory to detect recurrence and progression of the cancer.

Periodic cystoscopies are necessary to detect recurrence of transitional cell carcinoma (TCC). Patients with superficial bladder cancer usually have cystoscopy examinations every three months for the first year after diagnosis, every six-months for the next two years, and annually thereafter for the rest of their lives. If bladder cancer recurs, the regimen begins again (with treatment every 3 months for the first year, and so on). The reason for this regimen is that 60-70% of patients with superficial TCC have a recurrence of TCC and up to 40% suffer from progression to bladder/muscle-invasive TCC. Metastasis to a potentially life-threatening form of the cancer may occur if not diagnosed as early as possible.

The existing diagnostic as well as treatment apparatuses and methods are an enormous burden on patients. Continual invasive examinations take both a psychological as well as physical toll on the patients. There is thus a need to produce non-permanent cystoscopes, which are able to increase the rate of early detection of recurrent TCC and eliminate the need for repeated invasive procedures in the monitoring of TCC. Furthermore, there is a need to provide an apparatus and/or a method, which allows a cost effective monitoring of TCC.

PCT publication WO0054702, Yachia et al., discloses a system for treating a urinary bladder of an individual. U.S. Pat. No. 6,293,923, Yachia et al., discloses an expandable balloon for insertion into the urinary bladder of an individual. U.S. published application 2006/0189846, Huang et al., discloses a disposable two-step endoscope for examining human organs. U.S. published application 2007/0142711, Bayer et al., discloses an endoscope including a detachable wireless imaging device and an insertion tube having a distal end region. U.S. Pat. No. 7,251,383, Iddan et al., discloses an imager with a set of sensor elements and a fiber plate cover disposed on the set of sensor elements. U.S. Pat. No. 5,188,596, Condon et al., discloses a system for the dilation of the prostate urethra. PCT publication WO09426170, Companion et al., discloses a system for detecting, evaluating and treatment of prostate and urinary problems in a male human.

### SUMMARY OF THE DISCLOSURE

According to some embodiments there is provided a device for imaging a bladder that includes an image sensor, an illumination source and a device orientation actuator. The device may further be at least partially surrounded by a protective unit. The protective unit may include an expandable structural mesh.

According to some embodiments, the orientation actuator of the device may include a blast mechanism, a propeller, and the like, and may be used to control the orientation and positioning of the device in the surrounding in which it is situated.

According to some embodiments, the illumination source may include one or more illumination sources such as a light emitting diode (LED), an ultraviolet light source, an infrared light source, an incandescent light source or any combination thereof.

According to further embodiments, the device may further include an image sensor that comprises a digital camera. The digital camera may include, for example a CCD camera, a CMOS camera or the like.

According to some embodiments, there is provided an apparatus for delivering a self-contained bladder-imaging device to a bladder, wherein the apparatus comprises a handset. The apparatus may further include a cannula, an insertion element, a handle and any combination thereof.

According to some embodiments, there is provided a method for delivering a self-contained bladder imaging device to a bladder, that includes transporting a self-contained bladder imaging device through a urethra, using a handset, releasing said self-contained bladder imaging device into a bladder and extracting said handset from the urethra.

According to further embodiments, the bladder imaging device delivered by the method may be at least partially surrounded by a protective unit that may include an expandable structural mesh.

According to further embodiments, the bladder imaging device may further include an orientation actuator. The orientation actuator may include a ballast mechanism. The orientation actuator may include propeller.

According to further embodiments, the bladder imaging device may further include an illumination source. The illumination source may further include a light emitting diode (LED), an ultraviolet light source, an infrared light source, an incandescent light source or any combination thereof.

According to further embodiments, the bladder imaging device may further include an image sensor. The image sensor may include a digital camera. The digital camera may include, for example a CCD camera, CMOS camera, and the like.

According to some embodiments, the releasing of the self-contained bladder imaging device includes initiating an expansion.

### BRIEF DESCRIPTION OF THE FIGURES

Examples illustrative of embodiments of the invention are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 schematically illustrates an examination device, according to some embodiments;

FIG. 2 schematically illustrates an examination device and a protective unit, according to some embodiments;

FIG. 3 schematically illustrates an examination device and a protective unit, according to some embodiments;

FIG. 4 schematically illustrates an examination device and a protective unit, according to some embodiments;

FIG. 5 schematically illustrates a delivery apparatus, according to some embodiments;

FIG. 6 schematically illustrates a close-up view of a shielded examination device, according to some embodiments; and

FIG. 7 schematically illustrates a close-up view of a shielded examination device, according to some embodiments.

### DETAILED DESCRIPTION

According to some embodiments, there is provided a device that may be used for imaging a cavity of an internal organ having a cavity. The device may be inserted, non-permanently into a desired organ and may reside in the organ for any period of time. For example, the device may be used for imaging a bladder, uterus, stomach, and the like. The device may further include an image sensor, capable of obtaining images, for example, while being positioned inside the internal organ cavity. The device may further include an illumination source that may be used to illuminate the cavity of the internal organ. In addition, the device may comprise an orientation actuator that may be used to navigate the device inside the internal organ cavity by changing its spatial location inside the organ cavity. The examination device may be used to track, over an extended period of time, the internal cavity of the organ.

As referred to herein, the terms “imaging device”, “bladder imaging device”, “device”, “examination device” may interchangeably be used.

As referred to herein, the term “shielded examination device” may relate to a combination of an examination device and a protecting unit, wherein the examination device is at least partially surrounded by the protective unit.

As referred to herein, the term “user” may relate to health care providers, such as, a physician, a nurse, technician, and the like that may operate the examination device.

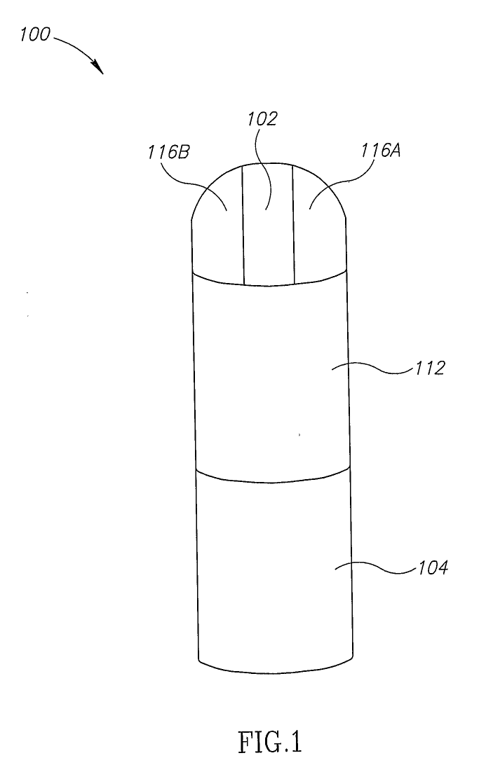

Reference is now made to FIG. 1, which schematically illustrates an imaging device, according to some embodiments. As shown in FIG. 1, a device, such as device 100, may have a cylinder-like shape and may include several functional subunits. For example, the device may include a power supply subunit, 104, that may be used to provide energy to the device in order to allow operation of the device. The device may further include an image sensor subunit, 102. The image sensor subunit may include, for example, a camera, that may be used to obtain images from the environment in which the device is situated. Device 100 may further include one or more illumination sources, such as illumination sources 116A-B. The Illumination source may be used to provide illumination of various forms and to illuminate the environment in which the device is located. In addition, device 100 may further include an orientation actuator subunit, such as orientation actuator 112. An orientation actuator, such as actuator 112 may be used to change the spatial orientation of the device within the environment in which it is located.

According to further embodiments, the imaging device may include additional subunits such as, for example, a logic device subunit that may include a processing unit. In addition, the device may include a receiver subunit. The receiver subunit may be used to receive information from a source that is external to the device. The device may further include a transmitter subunit that may be used to transmit information to a receiver that is external to the device. Information may include any type of data and/or instructions, such as, for example, digital data that may include for example, image data; electronic signals, and the like. Information transferred to the receiver subunit and information transferred from the transmitting unit may be transferred by any method of transferring information, such as wireless, by use of wires, and the like.

According to some embodiments, the imaging device may have any three dimensional shape. The device may be amorphous, cylindrical, cubical, ball-rounded, spherical, elongated or any other desired shape that may fit in size and function. The dimensions of the device may include any desired size that may be suitable to fit the cavity of the organ to which it is to be inserted. For example, the device may have a substantially cylindrical-like shape, with a diameter of, for example in the range of about 0.01 mm to 1.5 mm. For example, the diameter of the device may be in the range of about 0.01 mm-0.1 mm. For example, the diameter of the device may be in the range of about 0.1 mm-0.2 mm. For example, the diameter of the device may be in the range of about 0.2 mm-0.3 mm. For example, the diameter of the device may be in the range of about 0.3 mm-0.4 mm. For example, the diameter of the device may be in the range of about 0.4 mm-0.5 mm. For example, the diameter of the device may be in the range of about 0.5 mm-0.6 mm. For example, the diameter of the device may be in the range of about 0.6 mm-0.7 mm. For example, the diameter of the device may be in the range of about 0.7 mm-0.8 mm. For example, the diameter of the device may be in the range of about 0.8 mm-0.9 mm. For example, the diameter of the device may be in the range of about 0.9 mm-1 mm. For example, the diameter of the device may be in the range of about 1 mm-1.1 mm. For example, the diameter of the device may be in the range of about 1.1 mm-1.2 mm. For example, the diameter of the device may be in the range of about 1.2 mm-1.3 mm. For example, the diameter of the device may be in the range of about 1.3 mm to 1.5 mm. The length of the imaging device may be in the range of about 0.01 mm to 50 mm. For example, the length of the device may be in the range of about 0.01 to 0.1 mm. For example, the length of the device may be in the range of about 0.1 mm to 0.5 mm. For example, the length of the device may be in the range of about 0.5 mm to 1 mm. For example, the length of the device may be in the range of about 1 mm to 2 mm. For example, the length of the device may be in the range of about 2 mm to 5 mm. For example, the length of the device may be in the range of about 5 mm to 10 mm. For example, the length of the device may be in the range of about 10 mm to 20 mm. For example, the length of the device may be in the range of about 20 mm to 30 mm. For example, the length of the device may be in the range of about 30 mm to 40 mm. For example, the length of the device may be in the range of about 40 mm to 50 mm. The device may be comprised of various materials, such as gold, glass, ceramic, silicon, Teflon, polyethylene, any other biocompatible material, or any combination thereof.

According to some embodiments, the imaging device may include a power source subunit. The power source subunit may include one or more internal power sources that may include any kind of power source, such as, for example one or more rechargeable batteries that may include any type of rechargeable batteries that are known in the art, such as for example, but not limited to, Li-Ion, Ni—Me, Ni—Cd and the like. The power source may be recharged from an external source, such as, for example, by magnetic induction, electrical induction, and the like. The power source may also be recharged by an adaptable-wired connection to an external power source. The one or more power sources of the power source subunit may provide power/energy to the various other subunits of the examination device. According to some embodiments, some of the subunits of the examination device may have a respective power source, while other subunits may share a common power source. For example, the illumination source and the orientation actuator subunits may each have a respective power source, while other subunits may all receive power from a common power source.

According to some embodiments, the examination device may include an illumination subunit that may include one or more illumination sources. The illumination subunit may provide illumination of the surroundings in which the examination device is located. The illumination source may include any type of illumination source that is known in the art, such as, for example, Light emitting diode (LED), Organic light emitting diode (OLED), fluorescent light, optical fiber, Ultraviolet (UV) light source, Infrared (IR) light source, incandescent light source, light source that may emit light at a wavelength within the visual spectrum, and the like, or any combination thereof. The illumination provided by the illumination source may be predetermined, to comply with the environment which it is meant to illuminate and with the image sensor subunit which may need the illumination in order to capture viewable images. For example, an IR illumination source may be used with an image sensor subunit that is adapted to capture images at the IR spectrum. The illumination subunit may have a respective power source that may provide the energy needed to produce illumination. Activation of the illumination source may be controlled in various ways. For example, the activation of the illumination source may be automatic, such that the illumination source is activated at predetermined time intervals for a predetermined period of time. For example, the illumination source may illuminate for 3 seconds every 2 minutes. For example, the illumination source may be constitutively active and illuminate the organ cavity continuously. For example, activation of the illumination source may be coordinated with the image sensor subunit, such that when the image sensor subunit captures an image, that illumination source is activated. For example, the activation of the illumination source may be controlled by an external source, such as a user of the device. Controlling the illumination source by a user may be performed, for example, by transmitting a signal that is received by the receiving subunit of the examination device, processed by the logic device subunit and transferred to the illumination source.

According to further embodiments, the examination device may further include an image sensor subunit. The image sensor subunit may include any image sensitive element that may be used to capture images of the surroundings in which the imaging device is located. The image sensor element may include, for example, a digital camera, such as of the types: a charged coupled device (CCD), Complementary metal oxide semiconductor (CMOS), and the like. The image sensor may capture images at any frame rate and at any exposure time, meaning the images may include still images or video images. The frame rate and exposure time of the image sensor may be predetermined and/or may be controlled by the user. For example, the image sensor subunit may capture images at predetermined time intervals at a predetermined capture rate and predetermined exposure time. For example, the image sensor subunit may automatically capture still images at time intervals of about 2 minutes. For example, the image sensor may capture a series of consecutive images at a frame rate of 12 frames per second for a length of time of 5 minutes at time intervals of 10 minutes. According to further embodiments, capturing images by the image sensor may be controlled by a user. For example, a user may transmit a signal that may be received by the receiving subunit of the examination device, processed by the logic device subunit and transferred to the image sensor. Such activation of the image sensor by a user may be used to acquire still images or frames of images, for any length of time. The image sensor element may further include and/or be associated with a focusing element, such as one or more lenses. The lens may include various kinds of lenses, such as a compound lens, liquid lens, prismatic lens, a sensor lens and the like. The lens may further have various scopes and focal distances to allow better control of the images captured by the image sensor element. According to some embodiments, images captured by an image sensor may be viewed on-line in real time by a user, such as a health care provider (Physician, technician, nurse and the like). To this aim, the image sensor subunit may transmit the images acquired to the user. Transmission of the images acquired may be performed by aid of the transmitting subunit of the examination device. The transmitting subunit may transmit the image data acquired by the image sensor immediately as it has been acquired, to an external receiver. The external receiver that may be operated by a user, such as a health care provider, may further allow the user to view the acquired image data. According to further examples, the image data acquired by the image sensor is not transmitted to an external source but rather retained (stored) in the image sensor subunit. Retaining of the image data may be performed by use of any known storage method and any known memory media, such as, for example, use of memory chips, memory cards, and the like. The stored data may be retrieved by a user at a later time, for example, by transmission of the data to an external source, or by physical retrieval of the storage media.

According to additional embodiments, the examination device may further include an orientation actuator subunit. An orientation actuator may be used to change the spatial orientation and/or location of the examination device within the environment in which it is located. The orientation actuator may further be used to actively navigate the examination device within the cavity in which the examination device is located. Changing the orientation of the examination device may include changing its orientation along 360 degrees. Changing the location of the examination device may include changing the coordinates at which the examination device is located in the surroundings. The orientation actuator may include various types of orientation actuators, such as, but not limited to, use of an array of ballasts; one or more engines; an array of flaps and fins; an array of magnets, and the like, or any combination thereof. The orientation actuator may be controller by an external source, such as by a user (such as a health care professional) who may navigate the examination device in the environment in which the device is located. The user may transmit instructions to be received by the examination device receiver subunit. The instructions may be processed by the examination device logic device subunit that may then control the orientation actuator subunit. The orientation actuator subunit may be controlled automatically, for example, by predetermined operating instructions that may instruct the orientation actuator to position the examination device at a specified location at a specified time point at a specified orientation. According to some embodiments, the orientation actuator may include an array of ballasts. The array of ballasts may include one or more weights that may be situated in specialized partitions (compartments) within the examination device. The weights within the partitions may be located along various axes of the examination device. For example, a partition may be located along the vertical axis of the examination device, and an additional partition may be located along the horizontal axis of the examination device. The relative location of the weight situated in the vertical partition (vertical weight) and the weight situated in the horizontal partition (horizontal weight) may change the center of gravity of the examination device thereby allowing for a 360 degree freedom of movement of the examination device in the environment in which it is located. The location of the weights within their respective partitions along the respective axis may be actively controlled, for example, by an external user. For example, one or more motors may be used to move and change the location of the weights in their respective partitions. For example, when two motors are used, each motor can drive the weight in 2 directions in each of the respective axes (such as for example: +vertical axis; −vertical axis; +horizontal axis; −horizontal axis). Similarly, the orientation of the examination device may be controlled by one weight that may be situated in an elongated partition. The elongated partition may run along the vertical axis of the examination device, with the interior walls of the partition having spiral tracks, along which the weight may move. Changing the location of the weight along the spiral tracks may determine the orientation of the examination device within the cavity in which it is located. According to further embodiments, the examination device may include one or more engines that may be used to change the orientation and/or location of the examination device within a cavity in which it is located. The engines may include any type of engine that may fit in size and function. For example, the engine may include a piezoelectric engine, an electric engine, and the like. The engine may further be attached to a propelling means, such as, for example, propellers, that may be used to actively change the spatial location of the examination device. The engine may receive power from a respective power source. Operation of the engine may be controlled by an external source. For example, a user, such as a health care provider, may control activation of the engine and thus control the location of the examination device. In addition, the examination device may also include an array of fins (flaps). The array of fins may include one or more fins that may be located at various positions along the external surface of the examination device. The fins may include any surface, such as a flat surface, curved surface, and the like. The fins may be attached to the examination device in a manner that may allow at least a partial movement of the fins and may further allow changing the angle between the fins and the axes of the examination device. By changing the angle of the fins' surface relative to the examination device axes, the orientation of the examination device may change. For example, changing the angle of one or more fins of the examination device may change the orientation of the examination device. Movement of the fins may be controlled by a user, such as a health care provider. The user may change the orientation of the fins relative to the examination device and thus change its orientation. According to further embodiments, the orientation actuator may include ballast tanks. Ballast tanks may include one or more chambers that may be located at the surface of the examination device and may open/close to let external fluid inside the chambers. Filling the chambers may change the weight of the examination device and thus change its orientation. For example, three independent chambers may be located along the surface of the examination device. A user may determine which of the chambers to open, the extent of opening each chamber (and thus determine the amount of fluid entering the chamber and thus control the 360 degree orientation of the examination device. According to additional embodiments, the orientation actuator may include any combination of the orientation actuators described hereinabove.

According to further embodiments, the examination device may further include a logic device subunit. The logic device subunit may include a processing subunit, that may be used to control some or all subunits of the examination device. The logic device subunit may further include one or more integrated circuits (IC) or an arrangement of various kinds and numbers of controlling and storing devices, such as a microprocessor, a microcontroller, a random access memory (RAM), other memory storage media, and the like.

According to some embodiments, the examination device may further include an external protective unit. The protective unit may surround/cover at least a portion of the outer surface of the examination device. The protective unit may be used to aid in insertion of the examination device to reach its location, protect the examination device in the cavity of the organ in which it is located, aid in positioning the examination device, aid in the retrieval of the examination device, prevent the examination device from adhering to the internal walls of the organ, prevent unwanted exit of the examination device from the cavity of the organ, or any combination thereof. According to some embodiments, the protective unit may include various shapes and structures and may be comprised of various materials, such as metal wires, silicon, rubber, woven material, any other appropriate biocompatible material, and the like. The protective subunit may further be adapted to change in shape. For example, the protective unit may be collapsible, expandable, de-expandable (able to contract) and the like, so as to allow the transfer of the examination device through various openings of the body at various opening widths. Changing the shape/structure of the protective unit may be performed, for example, by a spring-like action that may hold the protective unit in a folded state, and when the spring is released, the structure may expand. Likewise, the protective unit may assume a deflated form and upon insertion into an organ cavity, fluids present in the cavity may inflate the protective unit. Likewise, the protective unit may be inflated by air, gases, and the like.

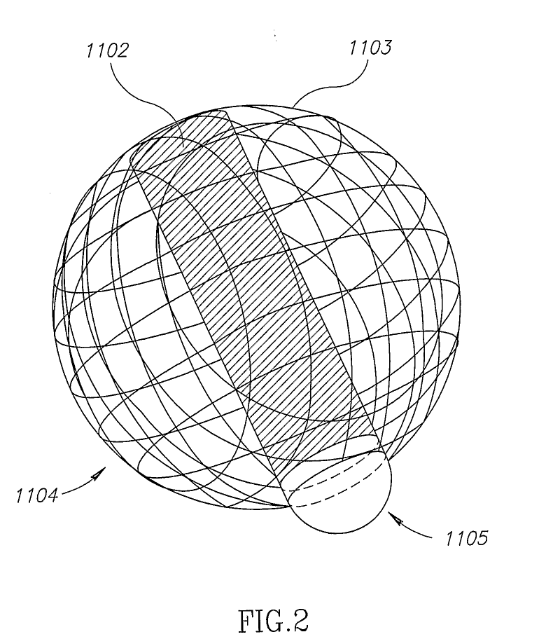

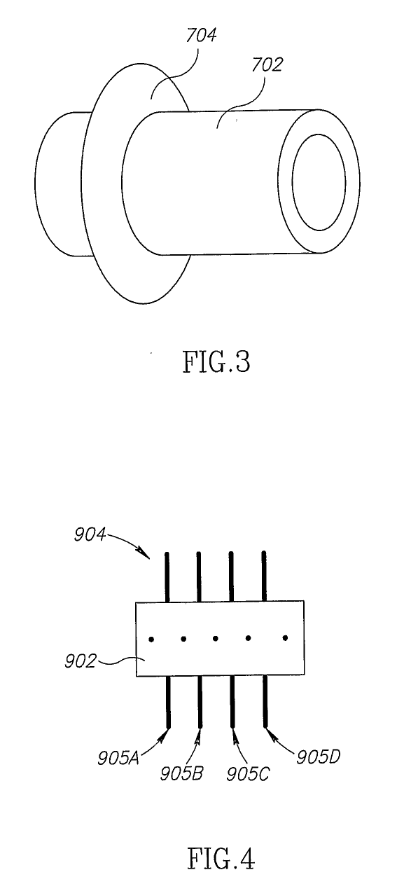

Reference is now made to FIG. 2, which illustrates an examination device with a protective unit, according to some embodiments. As shown in FIG. 2, an examination device, such as examination device 1102 may have a cylindrical shape. Encircling the examination device is protective unit 1104. The protective unit is shown in its expanded form, in a mesh-like form. As shown in FIG. 2, the protective unit is comprised of an array (mesh) of wires, such as wires 1103 that may include metal wires, silicon wires, and the like. The wires may be arranged in circles such that a substantially spherical structure is formed. The wires may be comprised of one wire that intertwines in circles or from several wires that are connected at their end. At one end of the examination device, an imaging subunit, such as imaging subunit 1105 is located. According to further embodiments, and as shown in FIG. 3, the protective unit may include a ring that encircles the examination device. An examination device, such as examination device 702 in FIG. 3 may have a cylindrical shape. A protective unit, such as protective unit 704 may have a substantially ring-like shape that may encircle the examination device. The protective unit may vary in width and may cover at least a portion of the examination device. Reference is now made to FIG. 4, which illustrates a protective unit, according to some embodiments. A protective unit, such as protective unit 904, may include a plurality of elements 905A-D. Each element 905A-D may be identical or different in length and broadness. Elements 905A-D of the protective subunit 904 may have any form, such as elliptical, hemispherical and the like. Examination device 902 may be attached to elements 905A-D of the protective unit 904. Examination device 902 may include recesses for some or all elements 905A-D of the protective unit 904.

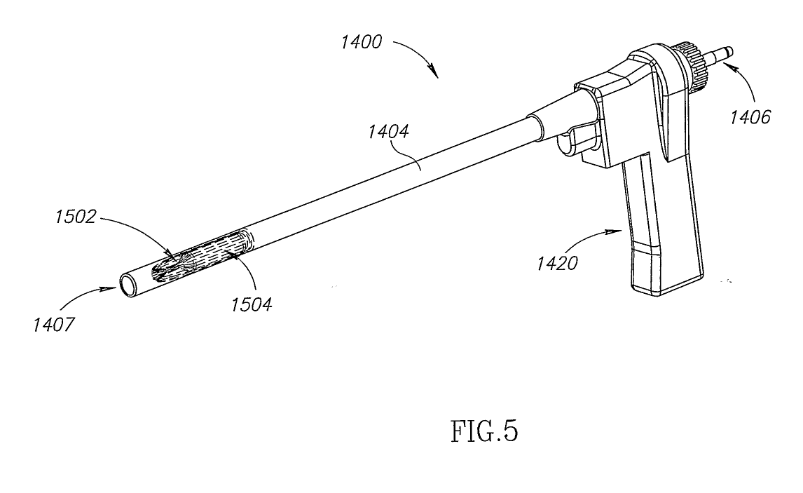

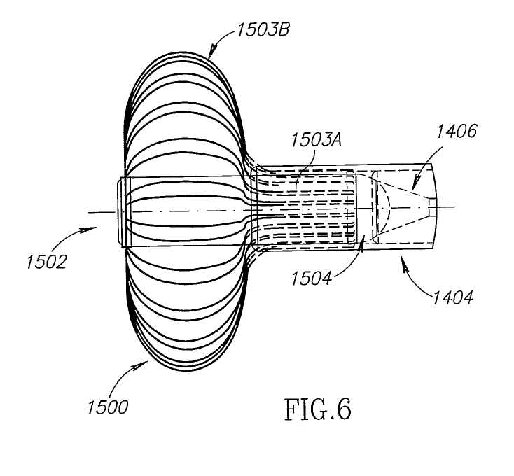

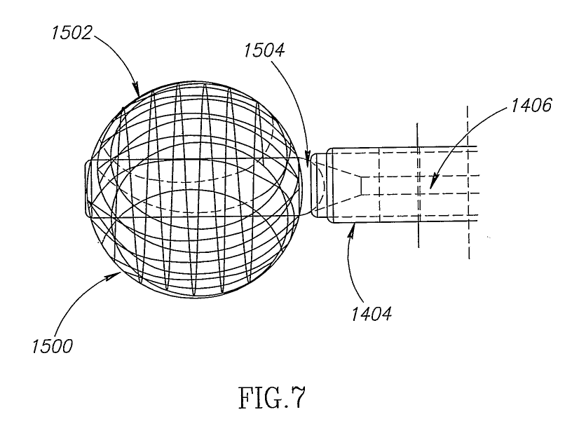

According to further embodiments, the examination device may be delivered to its location in the cavity of an internal organ such as a bladder by various ways. For example, the examination device may be inserted into a bladder using an endoscope, catheters, laparoscopic surgery, and the like. The examination device may further be delivered into the cavity of an internal organ by using an apparatus, such as delivery apparatus 1400 in FIG. 5, which illustrates such a delivery apparatus according to some embodiments. A delivery apparatus, such as delivery apparatus 1400 may include a handset. The delivery apparatus may include a grip (such as grip 1420). The delivery apparatus may further include a cannula, such as cannula 1404 that may be a hollow, flexible tube with an external diameter that may allow its passage through the urethra. For example, the external diameter of the cannula of the delivery apparatus may be in the range of about 0.01 mm to 1.5 mm. For example, the external diameter of the cannula may be in the range of about 0.01 mm-0.1 mm. For example, the external diameter of the cannula may be in the range of about 0.1 mm-0.2 mm. For example, the external diameter of the cannula may be in the range of about 0.2 mm-0.3 mm. For example, the external diameter of the cannula may be in the range of about 0.3 mm-0.4 mm. For example, the external diameter of the cannula may be in the range of about 0.4 mm-0.5 mm. For example, the external diameter of the cannula may be in the range of about 0.5 mm-0.6 mm. For example, the external diameter of the cannula may be in the range of about 0.6 mm-0.7 mm. For example, the external diameter of the cannula may be in the range of about 0.7 mm-0.8 mm. For example, the external diameter of the cannula may be in the range of about 0.8 mm-0.9 mm. For example, the external diameter of the cannula may be in the range of about 0.9 mm-1 mm. For example, the external diameter of the cannula may be in the range of about 1 mm-1.1 mm. For example, the external diameter of the cannula may be in the range of about 1.1 mm-1.2 mm. For example, the external diameter of the cannula may be in the range of about 1.2 mm-1.3 mm. For example, the external diameter of the cannula may be in the range of about 1.3 mm to 1.5 mm. The length of the cannula may be in the range of about 0.1 mm to 300 mm. For example, the length of the cannula may be in the range of about 0.1 to 10 mm. For example, the length of the cannula may be in the range of about 10 to 30 mm. For example, the length of the cannula may be in the range of about 30 to 60 mm. For example, the length of the cannula may be in the range of about 60 to 90 mm. For example, the length of the cannula may be in the range of about 90 to 120 mm. For example, the length of the cannula may be in the range of about 120 to 150 mm. For example, the length of the cannula may be in the range of about 150 to 180 mm. For example, the length of the cannula may be in the range of about 180 to 210 mm. For example, the length of the cannula may be in the range of about 210 to 240 mm. For example, the length of the cannula may be in the range of about 240 to 270 mm. For example, the length of the cannula may be in the range of about 270 to 300 mm. For example, the length of the cannula may be in the range of about 300 to 330 mm. For example, the length of the cannula may be in the range of about 330 to 360 mm. For example, the length of the cannula may be in the range of about 360 to 390 mm. For example, the length of the cannula may be in the range of about 390 to 420 mm. For example, the length of the cannula may be in the range of about 420 to 450 mm. For example, the length of the cannula may be in the range of about 450 to 480 mm. For example, the length of the cannula may be in the range of about 480 to 500 mm. The cannula may be attached on one end to the handset grip (1420). The opposing end of the cannula may be open to the environment (end 1407). At the open end (end 1407) of the cannula, an examination device (such as 1504), shielded by a protective unit (such as protective unit 1502) may be located. The protective unit (such as 1502) may be at its de-expandable form, such that the diameter of the shielded examination device is smaller than the internal diameter of the cannula. The shielded examination device may be located at any region along the internal space of the cannula. For example, as illustrated in FIG. 5, the shielded examination unit may be located inside the cannula, in close proximity to the open end of the cannula. The delivery apparatus may further include a positioning element (such as element 1406) that may be used to control the insertion and positioning of the shielded examination device to its location in the bladder. The positioning element may include an elongated tube that may be located inside the cannula. The positioning element may have a diameter that is smaller than the internal diameter of the cannula and may move freely within the inner region of the cannula. The positioning element may enter the cannula at the handset end and may be pushed towards the open end (1407) of the cannula. As the positioning element is pushed, it may in turn push the shielded examination device that is located inside the cannula towards the open end of the cannula. Thus, when the cannula is inserted into the urethra and the open end is located inside the bladder, the positioning element may be pushed and thus push the shielded examination device out of the cannula into the bladder cavity. As the shielded examination device is pushed out of the cannula, the protective unit may change form to an expanded form. FIG. 6 demonstrates a close up view of the open end of a cannula with a shielded examination device at least partially protruding outward of the cannula. As demonstrated in FIG. 6, the shielded examination device (such as shielded examination device, 1500) may be located at the open end of the cannula (such as cannula 1404). The shielded examination device may be at least partially inside the cannula wherein the protective unit (1502) is in de-expandable form (1503A); and at least partially outside of the cannula, wherein the protective unit is in expandable form (1503B). Also shown in FIG. 6 are an examination device (such as examination device 1504), and positioning element (such as element 1406). Reference is now made to FIG. 7, which schematically illustrates a close up view of the open end of a cannula with a shielded examination device released from the cannula. As demonstrated in FIG. 7, the shielded examination device (such as shielded examination device, 1504) may be located at the outer side of the open end of the cannula (such as cannula 1404). The shielded examination device may be released from the cannula with the protective unit (1502) at a fully expandable form. Also shown in FIG. 7 are an examination device (such as examination device 1504), and positioning element (such as element 1406). Once the shielded examination device is completely released from the inner portion of the cannula, the protective unit may be fully expanded. In this form, the shielded device may remain within the bladder cavity.

According to some embodiments, in order to retrieve the examination device from the cavity of the organ in which it is located, the protective unit may be de-expanded. De-expanding the protective unit may be performed in various ways. For example, when the protective unit is comprised of wires, such as in the form of a mesh and the like, one or more attachment points or springs on the protective unit may be engaged and opun pulling said spring/attachment points, the protective unit may collapse and de-expand. For example, when the protective unit is comprised of biocompatible material, the material may be a biodegradable material, and as such the protective unit may be degraded after a predetermined period of time has lapsed. Upon degradation of the protective unit, the examination device may be retrieved. For example, when the protective unit is comprised of inflatable material, the material may be deflated, for example, by puncturing the protective unit. Upon de-expanding the protective unit, the shielded examination device may be retrieved in various ways, such as for example, by using an apparatus that is similar to the positioning apparatus, that may be inserted into the cavity of the organ, which then attaches to the shielded examination device, and retrieves the examination device. Attachment of the positioning apparatus and the examination device may be enhanced by use of magnetic fields. For example, magnets may be present on the shielded examination device (on the protective unit and/or on the examination device) and on the positioning apparatus. The magnetic attraction between the magnets of the positioning device and the magnets of the shielded examination device may enhance the interaction and aid in retrieving the examination device from the cavity of the organ in which the examination device is located.

According to some embodiments, the interior of the examination device cavity may further include one or more inner compartments that may be used to store various substances, such as active substances, drugs, and the like. The inner compartments may further be adapted to open and release the substances into the environment. For example, the inner compartments may be adapted to slowly release, at predetermined time intervals the substances located within the inner compartments. In addition, the inner compartments may be adapted to receive fluids from the environment in which the device is positioned and to retain the fluids so that they may be examined at a later point in time by, for example, a health care provider.

According to some embodiments, the imaging device may also be used to provide surgical treatment inside the bladder. For example, the examination device may include a laser light source. The laser light source may be adapted to perform surgical procedures inside the bladder. For example, the examination device may include surgical tools, such as a scalpel that may be positioned on a pivoted arm, which is attached to the examination device. The surgical tool(s) may be operated by a user, such as a health care professional who may control the surgical tools, for example, by wireless communication routes.

According to some embodiments, the examination device may operate in a plurality of operational modes. For example, the examination device may have a sleep mode characterized by low energy consumption. For example, an active mode of the examination device may be an examination mode, in which one or more of the imaging device subunits are operative. For example, the light source may emit light, the image sensor may capture images, the orientation actuator may determine the orientation of the device, the logic device subunit may be operative, the receiving subunit may be operative, the transmitting subunit may be operative, or any combination thereof. In a randomized active mode, the examination device may be active at predetermined periods of time, for example, at 1 hour intervals the device may be operative for 20 minutes, over a time period of 24 hours.

According to some embodiments, the imaging device may be used to provide health care professionals and other users with a non-permanent examination device that may aid in examination of the inner cavity of various organs, such as the bladder, uterus, stomach, and the like. Upon insertion of the device to its location, the device may provide various types of data regarding the inner cavity of the organ. For example, the device may provide visual data regarding the inner cavity of the organ. Visual data may include individual images or consecutive frames of images acquired at various regions of the organ. The images may be acquired by the image sensor subunit, with the aid of lenses of various focal lengths that may aid in acquiring the best possible image. Visual images and image data of the organ may assist the health care professional in diagnostics and prognostics of medical conditions that are related to the organ. For example, image data may aid the health care professional in identification and tracking of tumors. The regions which may be examined are not limited to any particular zone of the organ. The user of the examination device may change the orientation and/or the spatial location of the examination device and thus determine the regions of interest to be examined. Changing the orientation and/or spatial location of the examination device may be performed by controlling the device orientation actuator. In addition, the device may further be used to provide other physiological data regarding the organ. For example, various sensors, such as a pH sensor, protein sensor, microbiological sensors, and the like may provide data regarding the physiological parameters of the organ. Such parameters may be related to various medical conditions inside the organ. Furthermore, the examination device may be used to deliver substances into the cavity of the organ. Such substances may include, for example, active substances, such as drugs, hormones, and the like. Such substances may be stored in inner compartments in the examination device and may be released at will, at predetermined time points, over an extended period of time, or any combination thereof.

The examination device may include an external control system, which may be used to control the examination device from an external location. For example, the external control system may be used to collect and/or assess and/or control and/or display and/or transmit and/or receive information, such as digital data, electrical signals, radio signals, and the like, to and from the examination device. Operation of the external control system may be performed by a health care professional, such as, for example, a physician, a nurse, a technician, and the like. The external control system may include several subunits, such as, for example, a controlling system, which may control and/or determine operation of the examination device. Operation of the examination device may include such parameters as: change of orientation; change of spatial location; change of mode of operation; illumination state; imaging state; receiving/transmitting state, and the like. The external control system may further include a transmitter subunit, and/or a receiver subunit that may be used to send and/or receive information to and from the examination device, respectively. The external control system may further include a logic device that may include a processor unit that may be able to process information received from and sent to the examination device. The external control system may further include a display screen that may display information received from the examination device, such as data, image data, and the like.

In the description and claims of the application, each of the words “comprise” “include” and “have”, and forms thereof, are not necessarily limited to members in a list with which the words may be associated.

The invention has been described using various detailed descriptions of embodiments thereof that are provided by way of example and are not intended to limit the scope of the invention. The described embodiments may comprise different features, not all of which are required in all embodiments of the invention. Some embodiments of the invention utilize only some of the features or possible combinations of the features. Variations of embodiments of the invention that are described and embodiments of the invention comprising different combinations of features noted in the described embodiments will occur to persons with skill in the art. It is intended that the scope of the invention be limited only by the claims and that the claims be interpreted to include all such variations and combinations.

1. A device for imaging a bladder, comprising:
an image sensor;
an illumination source; and
a device orientation actuator.

2. The device according to claim 1, at least partially surrounded by a protective unit.

3. The device according to claim 2, wherein said protective unit comprises a structural mesh.

4. The device according to claim 2, wherein said protective unit comprises an expandable structural mesh.

5. The device according to claim 1, wherein said device orientation actuator comprises a ballast mechanism.

6. The device according to claim 1, wherein said device orientation actuator comprises a propeller.

7. The device according to claim 1, wherein said illumination source comprises a light emitting diode (LED).

8. The device according to claim 1, wherein said illumination source comprises an ultraviolet light source.

9. The device according to claim 1, wherein said illumination source comprises an infrared light source.

10. The device according to claim 1, wherein said illumination source comprises an incandescent light source.

11. The device according to claim 1, wherein said image sensor comprises a digital camera.

12. An apparatus for delivering a self-contained bladder imaging device to a bladder, comprising a handset.

13. The apparatus according to claim 12, wherein the handset comprises a cannula.

14. The apparatus according to claim 12, wherein the handset comprises an insertion element.

15. The apparatus according to claim 12, wherein the handset comprises a handle.

16. A method for delivering a self-contained bladder imaging device to a bladder, comprising:
transporting a self-contained bladder imaging device through a urethra, using a handset;
releasing said self-contained bladder imaging device into a bladder; and
extracting said handset from the urethra.

17. The method according to claim 16, wherein said bladder imaging device is at least partially surrounded by a protective unit.

18. The method according to claim 17, wherein said protective unit comprises a structural mesh.

19. The method according to claim 17, wherein said protective unit comprises an expandable structural mesh.

20. The method according to claim 16, wherein said bladder imaging device comprises an orientation actuator.

21. The method according to claim 16, wherein said device orientation actuator comprises a ballast mechanism.

22. The method according to claim 16, wherein said device orientation actuator comprises a propeller.

23. The method according to claim 16, wherein said bladder imaging device comprises an illumination source.

24. The method according to claim 23, wherein said illumination source comprises a light emitting diode (LED).

25. The method according to claim 23, wherein said illumination source comprises an ultraviolet light source.

26. The method according to claim 23, wherein said illumination source comprises an infrared light source.

27. The method according to claim 23, wherein said illumination source comprises an incandescent light source.

28. The method according to claim 16 wherein said bladder imaging device further comprises an image sensor.

29. The method according to claim 28 wherein said image sensor comprises a digital camera.

30. The method according to claim 16, wherein the releasing of the self-contained bladder imaging device comprises initiating an expansion.
